(19) United States
(12) Patent Application Publication
(10) Pub. No.: US 2010/0203596 A1
(43) Pub. Date: Aug. 12, 2010

(54) HYBRIDIZATION CHAMBER FOR BIOASSAY AND HYBRIDIZATION METHOD USING THE HYBRIDIZATION CHAMBER

(75) Inventor: Byung-chul Kim, Suwon-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Gyeonggi-do (KR)

(21) Appl. No.: 12/545,401

(22) Filed: Aug. 21, 2009

(30) Foreign Application Priority Data
Feb. 9, 2009 (KR) 10-2009-0010205

(51) Int. Cl. C12P 19/34; C12M 1/00

(52) U.S. Cl. 435/91.2; 435/289.1

(57) ABSTRACT

Provided is a hybridization chamber comprising two biochips disposed in one chamber such that surfaces of the biochips, to which biomolecules that are to be analyzed are respectively bonded, face each other, reducing the distance between probe biomolecules, thereby reducing the distance along which biomolecules move in the hybridization chamber. Provided are also methods of hybridizing biomolecules using the hybridization chamber.

### CROSS-REFERENCE TO RELATED APPLICATIONS

This application claims priority to Korean Patent Application No. 10-2009-0010205, filed on Feb. 9, 2009, and all the benefits accruing therefrom under 35 U.S.C. §119, the contents of which in its entirety herein incorporated by reference.

### BACKGROUND

1. Field

Disclosed herein is a hybridization chamber for bioassay and a hybridization method using the hybridization chamber. More particularly, disclosed herein is a hybridization chamber for reducing the time for bioassay of an immobilized molecule and a target sample which is to be analyzed, and a hybridization method using the hybridization chamber.

2. Description of the Related Art

With entire DNA sequence of the human genome available, the interest in research on the function of the human genes, research on aspects of gene expression, on understanding of congenital diseases and cures thereof, and the development of new drugs has rapidly increased. Thus, there has been increasing demand for a method to rapidly analyze an enormous amount of genetic information with regard to the diagnosis, treatment, and prevention of hereditary diseases using biochips.

Biochips are devices for analyzing a biological substance, including bio-molecules such as enzymes, proteins, antibodies, deoxyribonucleic acid (DNA), and bio-organisms such as microorganisms, animal (e.g., nerve cells) and plant cells and components thereof. A biochip is formed by immobilizing on a support a probe to be analyzed with high density. The probe may be DNA, protein, antibodies, or the like. By detecting whether the probe is hybridized with a target material contained in a sample, genetic expression profile, genetic defects, protein distribution, reaction characteristics, or the like can be analyzed. Biochips are categorized into DNA chips, protein chips, and the like according to the type of probes used. For example, a DNA chip is formed by immobilizing a DNA oligomer on a solid substrate in a micro-array, and various DNA-based tests may be conducted using the DNA chip. In addition, biochips are categorized into micro-array chips affixed on solid supports and lab-on-a-chips affixed on micro-channels according to affixed subjects. Agitation and washing/drying systems are needed to attain effective hybridization between the target material contained in the sample, and the probe. When a sample is dropped on the biochip, only genes or proteins of the sample corresponding to a selected probe are bonded to the probe, and the remaining materials that are not bonded to the probe are washed away in a subsequent process. Accordingly, biometric information of a sample may be detected easily by testing to determine which probe of the biochip the sample is bonded to. Meanwhile, contrary to this, a biomolecule of a sample to be analyzed may be immobilized on a substrate in advance and then molecules of a reagent that is to react with the biomolecule may be dropped on the biomolecule.

However, for conventional biochips, the distance that molecules to be bonded to probes or samples immobilized on the substrate have to move until the molecules or samples are finally bonded is long, and thus a large amount of the molecules are required for bonding, and a long time is needed for bonding. Thus instead of letting the molecules move naturally through diffusion, a hybridization system which improves bonding between the molecules has been introduced. However, a long time for the hybridization is still needed. For example, in the case of a high density DNA micro-array chip, at least 16 hours is needed until the hybridization is completed.

In order to solve these problems, the inventor have developed an apparatus and method for non-specifically moving the molecules to a bonding area by applying electrical or magnetic force to the molecules.

### SUMMARY

Disclosed herein is a hybridization chamber for reducing the time for bioassay of immobilized molecules and a target sample which is to be analyzed, and a hybridization method using the hybridization chamber.

Additional aspects will be set forth in part in the description which follows and, in part, will be apparent from the description.

To achieve the above and/or other aspects, one or more embodiments includes a hybridization chamber comprising: a chamber wall defining a sealed space for hybridization; and a first biochip comprising a first probe surface; and a second biochip comprising a second probe surface, and a sample solution is provided between the first biochip and the second biochip, wherein the first biochip and the second biochip are disposed in parallel across two opposite inner surfaces of the chamber wall, and wherein the first biochip and the second biochip are disposed such that a first probe surface of the first biochip and a second probe surface of the second biochip, on which probe molecules are respectively formed, face each other.

In another embodiment, the hybridization chamber includes a first barrier that is disposed parallel to the first biochip and the second biochip between the first biochip and the second biochip across the two opposite inner surfaces of the chamber wall.

In another embodiment, the hybridization chamber includes a plurality of second barriers that are vertically mounted between the first and second probe surfaces of the first and second biochips, wherein the second barriers are perpendicular to the first and second probe surfaces of the first and second biochips so that a space between the first probe surface of the first biochip and the second probe surface of the second biochip is divided into a plurality of compartments.

In another embodiment, the hybridization chamber includes a cathode disposed near the first biochip and an anode disposed near the second biochip.

In one embodiment, the hybridization chamber further comprises target capture probe molecules immobilized on the first probe surface of the first biochip, and sample analyzing probe molecules are formed on the second probe surface of the second biochip. The target capture probe molecules and sample analyzing probe molecules that bond to the same biomolecules may be immobilized on corresponding opposite areas of the first probe surface and the second probe surface.

Sample analyzing probe molecules may be immobilized on both the first probe surface of the first biochip and the second probe surface of the second biochip.

The hybridization chamber may further include at least two spacers interposed between the first biochip and the second biochip so that a uniform distance is maintained between the first biochip and the second biochip.

The hybridization chamber may further include a reaction module defining an independent reaction space in the hybridization chamber, wherein the first and second biochips are disposed inside the reaction module and fixed.

The hybridization chamber may further include a first heating plate that is attached on inner surfaces of the chamber wall.

The hybridization chamber may further include a second heating plate that is disposed across the two opposite inner surfaces of the chamber wall and disposed near and parallel to the first biochip or the second biochip.

To achieve the above and/or other aspects, one or more embodiments includes a method of hybridizing biomolecules comprising disposing in a chamber a first biochip comprising a first probe surface, and a first barrier that is parallel to the first biochip; hybridizing a biomolecule to the first biochip by providing a sample solution comprising the biomolecule between the first biochip and the first barrier; disposing a second biochip comprising a second probe surface to be parallel to the first biochip, wherein the first biochip and the second biochip are disposed such that a first probe surface of the first biochip and a second probe surface of the second biochip face each other; removing the first barrier; and performing hybridization in the second biochip by moving biomolecules which are bonded to the first biochip, to the second biochip.

### BRIEF DESCRIPTION OF THE DRAWINGS

These and/or other aspects, advantages and features of the invention will become apparent by describing in further detail exemplary embodiments thereof with reference to the attached drawings, in which:

FIG. 1 is an exemplary schematic perspective view illustrating the configuration of a hybridization chamber;

FIG. 2 is an exemplary schematic cross-sectional view illustrating the hybridization chamber of FIG. 1;

FIG. 3 is an exemplary schematic cross-sectional view illustrating a hybridization chamber;

FIGS. 4A through 4C are exemplary schematic cross-sectional views illustrating a hybridization method;

FIG. 5 is an exemplary schematic cross-sectional view illustrating a hybridization chamber in which a plurality of barriers are disposed to divide a space between a first probe surface and a second probe surface into multiple compartments;

FIGS. 6A through 6C are exemplary schematic cross-sectional views illustrating a hybridization method; and

FIG. 7 is an exemplary schematic cross-sectional view illustrating a hybridization chamber in which spacers are inserted between a first biochip and a second biochip.

### DETAILED DESCRIPTION

Aspects, advantages, and features of the present invention and methods of accomplishing the same may be understood more readily by reference to the following detailed description of preferred embodiments and the accompanying drawings. The present invention may, however, may be embodied in many different forms, and should not be construed as being limited to the embodiments set forth herein. Rather, these embodiments are provided so that this disclosure will be thorough and complete and will fully convey the concept of the invention to those skilled in the art, and the present invention will only be defined by the appended claims.

It will be understood that when an element or layer is referred to as being “on” or “connected to” another element or layer, the element or layer can be directly on or connected to another element or layer or intervening elements or layers. In contrast, when an element or layer is referred to as being “directly on” or “directly connected to” another element or layer, there are no intervening elements or layers present. As used herein, the term “and/or” includes any and all combinations of one or more of the associated listed items.

It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer, or section from another region, layer or section. Thus, a first element, component, region, layer, or section discussed below could be termed a second element, component, region, layer, or section without departing from the teachings of the present invention.

Spatially relative terms, such as “below”, “lower”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “lower” relative to other elements or features would then be oriented “above” relative to the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a”, “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

In one embodiment, the hybridization chamber comprises a chamber wall defining a sealed space for hybridization; and a first biochip comprising a first probe surface; and a second biochip comprising a second probe surface, and a sample solution is provided between the first biochip and the second biochip, wherein the first biochip and the second biochip are disposed in parallel across two opposite inner surfaces of the chamber wall, and wherein the first biochip and the second biochip are disposed such that a first probe surface of the first biochip and a second probe surface of the second biochip, on which probe molecules are respectively formed, face each other.

In another embodiment, the hybridization chamber further comprises a first barrier that is disposed parallel to the first biochip and the second biochip between the first biochip and the second biochip across the two opposite inner surfaces of the chamber wall.

In another embodiment, the hybridization chamber further comprises a plurality of second barriers that are vertically mounted between the first and second probe surfaces of the first and second biochips, wherein the second barriers may be perpendicular to the first and second probe surfaces of the first and second biochips so that a space between the first probe surface of the first biochip and the second probe surface of the second biochip is divided into a plurality of compartments.

In another embodiment, the hybridization chamber further comprises a cathode disposed near the first biochip and an anode disposed near the second biochip.

In one embodiment, the hybridization chamber further comprises target capture probe molecules immobilized on the first probe surface of the first biochip, and sample analyzing probe molecules are formed on the second probe surface of the second biochip. Target capture probe molecules and sample analyzing probe molecules which bond to the same biomolecules may be immobilized on corresponding opposite areas of the first probe surface and the second probe surface.

Sample analyzing probe molecules may be immobilized on both the first probe surface of the first biochip and the second probe surface of the second biochip.

The hybridization chamber may further include at least two spacers interposed between the first biochip and the second biochip so that a uniform distance is maintained between the first biochip and the second biochip.

The hybridization chamber may further include a reaction module defining an independent reaction space in the hybridization chamber, wherein the first and second biochips are disposed inside the reaction module and fixed.

The hybridization chamber may further include a first heating plate that is attached on inner surfaces of the chamber wall.

The hybridization chamber may further include a second heating plate that is disposed across the two opposite inner surfaces of the chamber wall and disposed near and parallel to the first biochip or the second biochip.

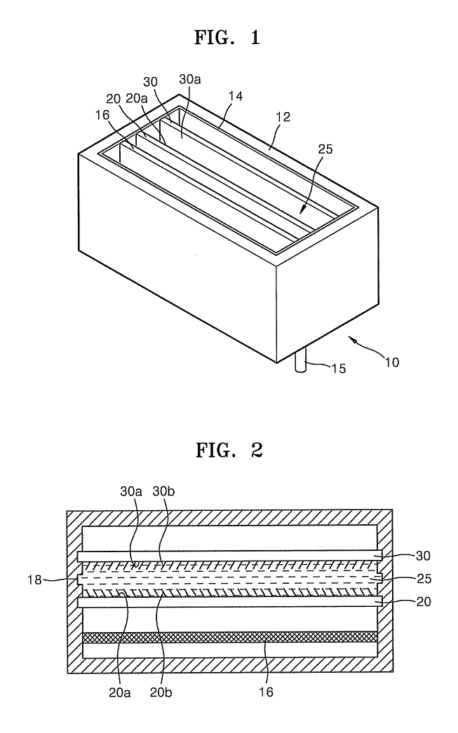

FIG. 1 is an exemplary schematic perspective view illustrating the configuration of a hybridization chamber 10. Referring to FIG. 1, the hybridization chamber 10 includes a chamber wall 12 defining a closed space for hybridization, and a first biochip 20 and a second biochip 30. The first biochip 20 and the second biochip 30 may be disposed parallel to each other across two opposite inner surfaces of the chamber wall 12 in the hybridization chamber 10. Also, the hybridization chamber 10 may include a first heating plate 14 that is attached on inner surfaces of the chamber wall 12 and a second heating plate 16 that is disposed across the two opposite inner surfaces of the chamber wall 12. As illustrated in FIG. 1, the first and second biochips 20 and 30 may be disposed parallel to the second heating plate 16. In FIG. 1, the first biochip 20 is disposed parallel and adjacent to the second heating plate 16. However, the second biochip 30 may also be disposed adjacent to the second heating plate 16.

In one embodiment, the first biochip 20 and the second biochip 30 are disposed such that surfaces thereof, on which probe molecules are formed, hereinafter referred to as probe surfaces, face each other. For example, as illustrated in FIG. 1, a first probe surface 20a of the first biochip 20 faces a second probe surface 30a of the second biochip 30. The distance between the a first probe surface 20a of the first biochip 20 and the a second probe surface 30a of the second biochip 30 is less than about 1 cm, less than about 100 μm, less than about 10 μm, less than about 1 μm, less than about 0.5 μm. In one embodiment, the first heating plate 14 and the second heating plate 16 are controlled separately and control temperature independent of the other. In this exemplary configuration, the first heating plate 14 may control the overall temperature inside the hybridization chamber 10, and the second heating plate 16 may control temperatures of the first biochip 20 and the second biochip 30 which are disposed near the second heating plate 16. Accordingly, when the first heating plate 14 and the second heating plate 16 are controlled independently, a wide temperature range may be controlled using the first heating plate 14, and a narrower temperature range may be controlled using the second heating plate 16. In an alternative embodiment, the first heating plate 14 and the second heating plate 16 may contact each other and control temperature in cooperation. In this case, a reaction temperature in a space surrounded by the first heating plate 14 and the second heating plate 16 may be maintained uniformly.

As illustrated in FIG. 1, an upper surface of the hybridization chamber 10 may be left open. Alternatively the space inside the hybridization chamber 10 may be completely sealed using an upper cover (not shown). When the upper cover is used, a uniform temperature during reaction may be easily maintained and evaporation of a reaction sample solution can be minimized. Also, another heating plate (not shown) may be formed on an inner surface of the upper cover.

In one embodiment, the hybridization chamber 10 further includes an outlet 15 further formed under the hybridization chamber 10, for discharging a reaction sample solution or a washing solution.

Referring to FIG. 2, the second heating plate 16 and the first and second biochips 20 and 30 are disposed parallel to one another in the hybridization chamber 10. For convenience, the first heating plate 14 is not illustrated in FIG. 2. The first and second biochips 20 and 30 may be inserted into grooves 18 that are vertically formed in the inner surface of the chamber wall 12 and fixed. Also, grooves 18 may be further formed in the chamber wall 12 between the first biochip 20 and the second biochip 30 so that a barrier, which will be described later, is inserted into the grooves 18 and immobilized. In FIGS. 1 and 2, a set of the second heating plate 16 and the first and second biochips 20 and 30 are illustrated. Alternatively, a plurality of sets of the second heating plates 16 and the first and second biochips 20 and 30 may be disposed in parallel in the hybridization chamber 10 according to necessity.

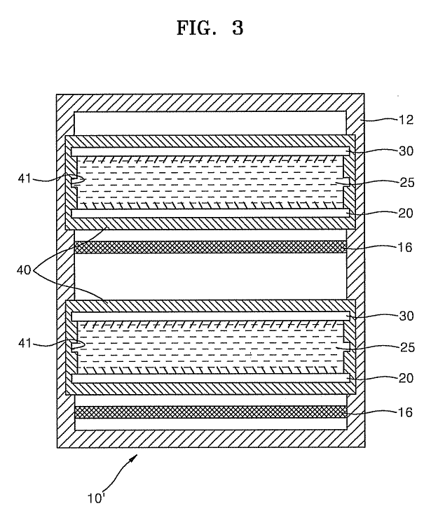

In one embodiment, a reaction module 40 that secures each of the sets of the first and second biochips 20 and 30 may be used when the sets of the second heating plates 16 and the first and second biochips 20 and 30 are disposed in the hybridization chamber 10, as illustrated in FIG. 3. Referring to FIG. 3, two sets of the second heating plates 16 and the first and second biochips 20 and 30 are installed in the hybridization chamber 10. Here, the first and second biochips 20 and 30 of each set are secured by reaction modules 40, respectively, and the reaction modules 40 may be disposed in the hybridization chamber 10 adjacent to their corresponding second heating plates 16, respectively. Grooves 41 may be formed vertically in an inner wall of the reaction module 40 so that the first and second biochips 20 and 30 are inserted and fixed. Also, grooves 41 may be further formed in an inner wall of the reaction module 40 between the first biochip 20 and the second biochip 30 so that a barrier, which will be described later, may be inserted and fixed. By using the reaction module 40, a separate reaction space from other reaction spaces of the first and second biochips 20 and 30 of other sets of the hybridization chamber 10 may be formed.

As described above, the first biochip 20 and the second biochip 30 are disposed such that the first probe surface 20a of the first biochip 20 and the second probe surface 30a of the second biochip 30 face each other. Referring to FIG. 2, the first probe surface 20a, on which a plurality of first probe molecules 20b are arranged, and the second probe surface 30a, on which a plurality of second probe molecules 30b are arranged, face each other. In this configuration, a sample solution 25 for reacting with the first probe molecules 20b and/or the second probe molecules 30b is provided between the first biochip 20 and the second biochip 30. Accordingly, a reaction space is formed between the first biochip 20 and the second biochip 30.

Various benefits are obtained by disposing the first biochip 20 and the second biochip 30 such that the first probe surface 20a and the second probe surface 30a face other. For example, target capture probe molecules may be arranged on the first probe surface 20a of the first biochip 20 as the first probe molecules 20b so that the first probe molecules 20b quickly bond with biomolecules in the sample solution 25, or the bonded biomolecules may be amplified. Further, sample analyzing probe molecules which are generally used in sample analysis may be densely arranged on the second probe surface 30a of the second biochip 30. When probe molecules that are to be bonded with the same biomolecules are respectively arranged in opposite areas of the first probe surface 20a and the second probe surface 30a, a path along which the biomolecules in the sample solution 25 move may be shortened. Accordingly, when the biomolecules in the sample solution 25 which are to be analyzed are bonded with the first biochip 20 in advance, the time needed for hybridization in the second biochip 30 used in an actual sample analysis may be reduced.

In another embodiment, the first biochip 20 and the second biochip 30 may be independent from each other. For example, different probe molecules may be arranged on the first probe surface 20a and the second probe surface 30a, respectively, and both the first biochip 20 and the second biochip 30 may be used in the actual sample analysis. Also, for example, the first biochip 20 may be a ribonucleic acid (RNA) chip used in the sample analysis, and the second biochip 30 may be a DNA chip also used in a sample analysis. In this case, one sample may be analyzed from various aspects. The first biochip 20 and the second biochip 20 may be the same or different according to the bioassay. In this case where the first biochip 20 and the second biochip 20 may be the same, the accuracy of the sample analysis may be further increased.

Further disclosed herein are hybridization method using the hybridization chamber. In one embodiment, the hybridization method disposing in a chamber a first biochip comprising a first probe surface, and a first barrier that is parallel to the first biochip; hybridizing a biomolecule to the first biochip by providing a sample solution comprising the biomolecule between the first biochip and the first barrier; disposing a second biochip comprising a second probe surface to be parallel to the first biochip, wherein the first biochip and the second biochip are disposed such that a first probe surface of the first biochip and a second probe surface of the second biochip face each other; removing the first barrier; and performing hybridization in the second biochip by moving biomolecules which are bonded to the first biochip, to the second biochip.

Target capture probe molecules may be formed on the first probe surface of the first biochip, and sample analyzing probe molecules may be formed on the second probe surface of the second biochip.

The target capture probe molecules and sample analyzing probe molecules that respectively bond to the same biomolecules may be formed on corresponding opposite areas of the first probe surface and the second probe surface.

The performing of hybridization in the second biochip may include: disposing a cathode near the first biochip and an anode near the second biochip to form an electrical field in a direction from the second biochip to the first biochip; moving the biomolecules bonded to the first probe surface to the second probe surface using an electrophoresis method; installing the first barrier again between the first biochip and the second biochip; and catalyzing the hybridization in the second biochip by adjusting a reaction temperature.

The performing of hybridization in the second biochip may include: installing a plurality of second barriers between the first and second probe surfaces of the first and second biochips to be perpendicular to the first and second probe surfaces of the first and second biochips so as to divide a space between the first biochip and the second biochip into a plurality of compartments; cutting the biomolecules bonded to the first probe surface using a restriction enzyme; and catalyzing the hybridization in the second biochip by adjusting a reaction temperature.

Restriction sites may be formed in the biomolecules in the sample solution in advance.

The method may further include, after performing the hybridization in the first biochip and removing the first barrier, connecting terminals of the biomolecules attached to probe molecules formed on the first probe surface and terminals of the probe molecules formed on the first probe surface, using ligase.

The performing of the hybridization in the second biochip may include: adjusting a distance between the first biochip and the second biochip to be less than 1 μm; and allowing the biomolecules to be disjoined from the probe molecules formed on the first probe surface and be bonded to the probe molecules formed on the second probe surface by adjusting a reaction temperature, wherein the biomolecules are continuously connected to the terminals of the probe molecules formed on the first probe surface after being disjoined from the probe molecules formed on the first probe surface.

The method may further include, after performing the hybridization in the first biochip and before removing the first barrier, amplifying the biomolecules hybridized on the first biochip.

The amplifying of the biomolecules may be performed using a polymerase chain reaction (PCR) method using polymerase, and the method may further include performing a fluorescent labeling process on the amplified biomolecules.

Hereinafter, various examples of hybridization and sample analysis methods using the above-described hybridization chamber 10 will be described. In the following examples, for convenience of description, the first and second biochips 20 and 30 and a barrier 13 are described as being directly installed on the chamber wall 12. However, the first and second biochips 20 and 30 and a barrier 13 may be directly installed on the reaction module 40 illustrated in FIG. 3.

### HYBRIDIZATION EXAMPLE 1

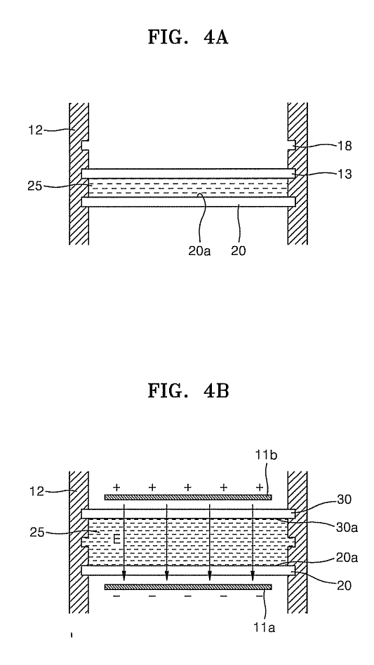

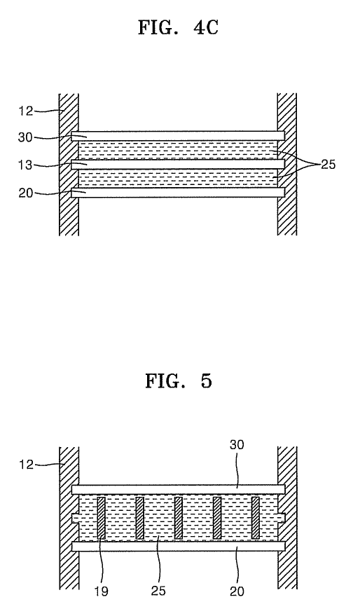

For this example, the hybridization and sample analysis method of biomolecules using the above-described hybridization chamber 10 will be described with reference to FIGS. 4A to 4C. First, as illustrated in FIG. 4A, a first biochip 20 and a barrier 13 that is parallel to the first biochip 20 are installed in the grooves 18 of the chamber wall 12 and fixed. In FIG. 4A, a portion of the hybridization chamber 10 is illustrated for convenience. A target capture DNA micro-array which bonds with a particular biomolecule of a sample is immobilized on a first probe surface 20a of the first biochip 20. Then, a sample solution 25 is added the space between the first biochip 20 and the barrier 13. For this example, the sample solution 25 includes biomolecules a fluorescent-marked human full-length DNA. Following the addition of sample solution 25 to the space between the first biochip 20 and the barrier 13, the biomolecules in the sample solution 25 are selectively bonded on the first probe surface 20a of the first biochip 20 through a hybridization reaction.

Next, as illustrated in FIG. 4B, the second biochip 30 is installed on the chamber wall 12 parallel to the first biochip 20 and fixed, and then barrier 13 is removed. Alternatively, the second biochip 30 may be installed in advance in the above operation described with respect to FIG. 4A. The second biochip 30 is positioned such that the second probe surface 30a of the second biochip 30 directly faces the first probe surface 20a of the first biochip 20. Thus, when the barrier 13 is removed, the second probe surface 30a of the second biochip 30 directly faces the first probe surface 20a of the first biochip 20, having the sample solution 25 therebetween. The distance between the first biochip 20 and the second biochip 30 may be, for example, about 1 cm. For this example, a DNA micro-array for genotyping of the human full-length DNA is formed on the second probe surface 30a. In order that DNA micro-arrays corresponding to one another are respectively arranged on opposite areas of the first probe surface 20a and the second probe surface 30a, the first biochip 20 and the second biochip 30 may be manufactured in advance. In this case, the positions of the first biochip 20 and the second biochip 30 may be aligned in advance so that a distance between the opposite areas of the first probe surface 20a and the second probe surface 30a is minimized to be the shortest possible distance. Alternatively, the second biochip 30 may be aligned after the biomolecules are bonded to the first probe surface 20a so that the distance between opposite areas of the first probe surface 20a and the second probe surface 30a is minimized to be the shortest possible distance.

Following the installation of the second biochip 30 on the chamber wall 12 parallel to the first biochip 20 and removal of barrier 13, a cathode 11a is disposed near the first biochip 20 and an anode 11b is disposed near the second biochip 30 to apply a current, as illustrated in FIG. 4B. Accordingly, an electrical field in a direction from the second biochip 30 to the first biochip 20 is formed across the first and second biochips 20 and 30 and the sample solution 25. Since the biomolecules (DNA molecules) that are bonded to the first probe surface 20a have (−) charges, they move to the second probe surface 30a in a reverse direction of the electrical field by electrophoresis.

After the biomolecules have sufficiently moved to the second probe surface 30a, the barrier 13 is installed again between the first biochip 20 and the second biochip 30 as illustrated in FIG. 4C so that the biomolecules do not return to the first probe surface 20a. Then, when hybridization is catalyzed by controlling the reaction temperature, etc., the biomolecules bond on the second probe surface 30a. The biomolecules in the sample solution 25 are then selectively bonded on the second probe surface 30a of the second biochip 30 through a hybridization reaction. After the hybridization is completed in the second biochip 30, the second probe surface 30a may be scanned using a well-known fluorescence scanning method to analyze a sample.

According to the above-described method, hybridization in the second biochip 30 may be quickly performed by finishing hybridization in the first biochip 20 in advance, positioning the second biochip 30 for the actual sample analysis to directly face the first biochip 20.

### HYBRIDIZATION EXAMPLE 2

In this example, biomolecules bonded to the first probe surface 20a may be bonded to the second probe surface 30a without using an electrophoresis method. Thus, for this example, hybridization in the first biochip 20 is performed as described above with reference to FIG. 4A. Specifically, as illustrated in FIG. 4A, a first biochip 20 and a barrier 13 that is parallel to the first biochip 20 are installed in the grooves 18 of the chamber wall 12 and fixed. In FIG. 4A, a portion of the hybridization chamber 10 is illustrated for convenience. A target capture DNA micro-array which bonds with a particular biomolecule of a sample is formed immobilized on a first probe surface 20a of the first biochip 20. Then, a sample solution 25 is added the space between the first biochip 20 and the barrier 13. For this example, the sample solution 25 includes biomolecules including a fluorescent-marked human full-length DNA may be present in the sample solution 25. Following the addition of sample solution 25 to the space between the first biochip 20 and the barrier 13, the biomolecules in the sample solution 25 are selectively bonded on the first probe surface 20a of the first biochip 20 through a hybridization reaction.

Next, the second biochip 30 is installed on the chamber wall 12 and fixed, and then barrier 13 is removed. As described above, the position of the second biochip 30 may be aligned such that a distance between corresponding areas of the first probe surface 20a and the second probe surface 30a is minimized to be the shortest possible distance. Next, as illustrated in FIG. 5, a plurality of barriers 19 for dividing the space between the first probe surface 20a and the second probe surface 30a into multiple compartments are installed. The barriers 19 are installed perpendicularly to the first probe surface 20a and the second probe surface 30a. For example, a plurality of grooves (not shown) may be formed in a bottom surface of the hybridization chamber 10 to install the barriers 19. Alternatively, the barriers 19 may be fixed on the upper cover (not shown) of the hybridization chamber 10. Thus, biomolecules may move only in areas surrounded by the barriers 19. Accordingly, the distance the biomolecules move until they are finally bonded to the second probe surface 30a may be minimized.

Once the barriers 19 have been installed, the DNA double helixes of the human full-length DNA hybridized on the first probe surface 20a are cut using a restriction enzyme, such as, for example Hinf1. To this end, restriction sites may be formed in advance on the DNA double helixes of the sample to be analyzed. Next, the hybridization of the sample separated from the first probe surface 20a by the restriction enzyme to the second probe surface 30a is facilitated by adjusting the temperature. When the hybridization in the second biochip 30 is complete, the second probe surface 30a is scanned using a well-known fluorescence scanning method to analyze the sample.

### HYBRIDIZATION EXAMPLE 3

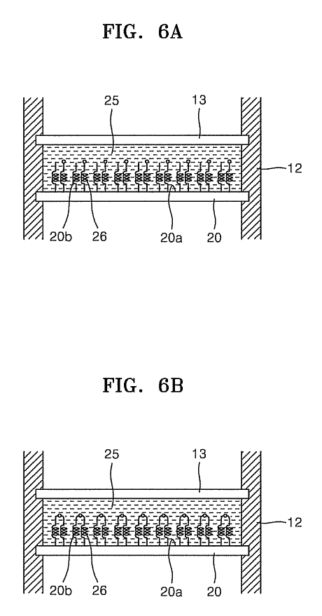

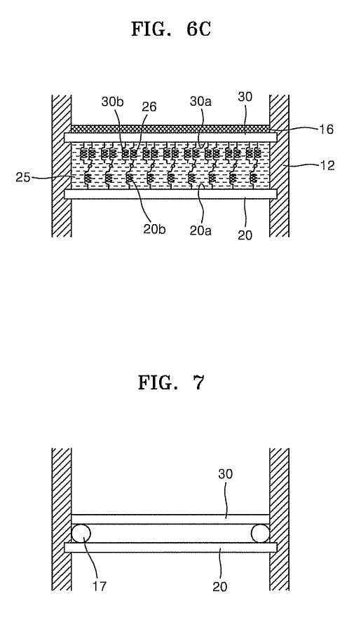

In this example, the distance between the opposite areas of the first probe surface 20a and the second probe surface 30a is minimized further, such that the distance the biomolecules hybridized on the first probe surface 20a move may be further shortened. For this example, first, hybridization in the first biochip 20 is performed as described with reference to FIG. 4A. Specifically, as illustrated in FIG. 4A, the first biochip 20 and the barrier 13 that is parallel to the first biochip 20 are installed in the grooves 18 of the chamber wall 12 and fixed. FIG. 6A illustrates, in exaggeration, the hybridization in the first biochip 20. For this example, a plurality of the first probe molecules 20b, specifically, DNA oligomers, are arranged on the first probe surface 20a of the first biochip 20. Then, a sample solution 25 is added the space, wherein the sample solution 25 includes biomolecules 26 capable of hybridizing to the plurality of the first probe molecules 20b. The biomolecules 26 of a sample are bonded with the first probe molecules 20b through a hybridization reaction. The rest of the biomolecules 26 which are not bonded with the first probe molecules 20b may obviously remain in the sample solution 25 but are not illustrated.

Then, the terminal end of biomolecules 26 bonded are ligated to the first probe molecules 20b using ligase, such as, for example, T4 DNA ligase. Thus, the terminal end of the two DNA molecules are connected to each other. That is, a terminal end of the biomolecules 26 which are attached to the first probe molecules 20b and a terminal of the first probe molecules 20b are connected to each other due to ligase treatment as illustrated in FIG. 6B. FIG. 6B illustrates in exaggeration two DNA molecules (e.g. first probe molecules 20b and biomolecules 26) connected to each other following ligation using ligase.

Next, as described above, the second biochip 30 is installed in the hybridization chamber 10 and fixed, and then barrier 13 is removed. Here, the position of the second biochip 30 may be aligned such that the distance between corresponding areas of the first probe surface 20a and the second probe surface 30a is minimized to be the shortest possible distance, as is described above. For example, the distance between the first biochip 20 and the second biochip 30 may be adjusted to be less than about 1 μm. In order to arrange the first biochip 20 and the second biochip 30 as close to each other as possible, spacers 17 having a diameter of about 1 μm or smaller may be interposed between the first biochip 20 and the second biochip 30 and the second biochip 30 may be pressurized toward the first biochip 20, as shown in FIG. 7. By using the spacer 17, a uniform distance of about 1 μm or less between the first biochip 20 and the second biochip 30 may be easily maintained.

After barrier 13 has been removed, the biomolecules 26 connected to the terminal ends of the first probe molecules 20b may be hybridized on the second probe surface 30a, by catalyzing hybridization by, for example, adjusting the temperature. FIG. 6C illustrates, in exaggeration, the biomolecules 26 of about 500-base to about 5000-bases in length that are hybridized on the second probe surface 30a, on which DNA molecules of about 20-base to about 70-base are formed. That is, as shown in FIG. 6C, the biomolecules 26 are first denatured from the first probe molecules 20b and are then bonded to the second probe molecules 30b through a hybridization reaction. According to this example, the terminal ends of biomolecules 26 are connected to the terminals of the first probe molecules 20b, and thus the barrier 19 as illustrated in FIG. 5 need not be used. In the hybridization process illustrated in FIG. 6C, when the sample is heated to about 95° C. using the second heating plate 16, the biomolecules 26 separate from the first probe molecules 20b. Then, the sample is cooled to about 4° C. using the second heating plate 16, and the biomolecules 26, which are no longer hybridized to the first probe molecules 20b, and which are still ligated to the terminal ends of the first probe molecules 20b, are then bonded to their corresponding second probe molecules 30b. Accordingly, the biomolecules 26 do not float in the sample solution 25 and thus the hybridization in the second biochip 30 may be performed more quickly and more reliably.

Then the second biochip 30 is be removed from the hybridization chamber 10 for additional sample analysis. In this case, the biomolecules 26 are completely separated from the first probe molecules 20b but remain bonded to the second probe molecules 30b. As the hybridization in the second biochip 30 is completed, the second probe surface 30a is scanned using a well-known fluorescence scanning method to analyze the sample.

### HYBRIDIZATION EXAMPLE 4

In this example, the biomolecules of the sample hybridized on the first biochip 20 are be amplified. First, hybridization in the first biochip 20 is performed as described with reference to FIG. 4A. Specifically, as illustrated in FIG. 4A, the first biochip 20 and the barrier 13 that is parallel to the first biochip 20 are installed in the grooves 18 of the chamber wall 12 and fixed. Then a DNA sample that is hybridized on the first biochip 20. The DNA sample is then amplified using any well-known nucleic acid amplification method, such as, a polymerase chain reaction (PCR) method or a rolling circle amplification (RCA) method. For example, DNA is be synthesized by using the hybridized DNA sample as a template and using a polymerase such as a thermostable DNA polymerase or phi29 DNA polymerase. Then fluorescent labeling is performed on the synthesized DNA using a well-known method.

Then the amplified biomolecules of the sample hybridized on the first probe surface 20a of the first biochip 20 are then hybridized on the second probe surface 30a of the second biochip 30. The hybridization in the second biochip 30 may be performed using the method described with reference to Hybridization Example 1 or 2.

For example, the second biochip 30 may be installed on the in the hybridization chamber 10 on the chamber wall 12 and fixed, and then barrier 13 is removed. Next the amplified biomolecules are directed toward the second biochip 30 using an electrophoresis method illustrated in FIG. 4B. Then as illustrated in FIG. 4C, the barrier 13 may be installed again and the reaction temperature may be adjusted to catalyze the hybridization. Alternatively, as illustrated in FIG. 5, a plurality of barriers 19 which enable dividing a space between the first probe surface 20a and the second probe surface 30a into multiple compartments may be installed. Once the barriers 19 have been installed, the amplified biomolecules may be cut using a restriction enzyme, such as, for example, Hinf1. Next, the hybridization of the sample separated from the first probe surface 20a by the restriction enzyme to the second probe surface 30a is facilitated by adjusting the reaction temperature.

As the sample is amplified on the first biochip 20, the processes of amplifying an extracted sample and hybridizing the amplified sample in the second biochip 30 may be performed continuously in one hybridization chamber 10. Accordingly, the processes of amplifying a sample through analyzing the sample may be conducted as one procedure.

It should be understood that the exemplary embodiments described therein should be considered in a descriptive sense only and not for purposes of limitation. Descriptions of features or aspects within each embodiment should typically be considered as available for other similar features or aspects in other embodiments.

What is claimed is:

1. A hybridization chamber comprising:
a chamber wall defining a sealed space for hybridization; and
a first biochip comprising a first probe surface; and
a second biochip comprising a second probe surface, and
a sample solution is provided between the first biochip and the second biochip,
wherein the first biochip and the second biochip are disposed in parallel across two opposite inner surfaces of the chamber wall, and
wherein the first biochip and the second biochip are disposed such that a first probe surface of the first biochip and a second probe surface of the second biochip, on which probe molecules are respectively formed, face each other.

2. The hybridization chamber of claim 1, further comprising a first barrier disposed parallel to the first biochip and the second biochip and between the first biochip and the second biochip across the two opposite inner surfaces of the chamber wall.

3. The hybridization chamber of claim 1, further comprising a plurality of second barriers that are vertically mounted between the first and second probe surfaces of the first and second biochips,
wherein the second barriers are perpendicular to the first and second probe surfaces of the first and second biochips so that a space between the first probe surface of the first biochip and the second probe surface of the second biochip is divided into a plurality of compartments.

4. The hybridization chamber of claim 1, further comprising a cathode disposed near the first biochip and an anode disposed near the second biochip.

5. The hybridization chamber of claim 1, further comprising target capture probe molecules immobilized on the first probe surface of the first biochip, and sample analyzing probe molecules immobilized on the second probe surface of the second biochip.

6. The hybridization chamber of claim 5, wherein the target capture probe molecules and sample analyzing probe molecules bond to the same biomolecules and are formed on corresponding opposite areas of the first probe surface and the second probe surface.

7. The hybridization chamber of claim 1, wherein sample analyzing probe molecules are immobilized on both the first probe surface of the first biochip and the second probe surface of the second biochip.

8. The hybridization chamber of claim 1, further comprising at least two spacers interposed between the first biochip and the second biochip so that a uniform distance is maintained between the first biochip and the second biochip.

9. The hybridization chamber of claim 1, further comprising a reaction module defining an independent reaction space in the hybridization chamber, wherein the first and second biochips are disposed inside the reaction module and fixed.

10. The hybridization chamber of claim 1, further comprising a first heating plate that is attached on inner surfaces of the chamber wall.

11. The hybridization chamber of claim 1, further comprising a second heating plate that is disposed across the two opposite inner surfaces of the chamber wall and disposed near and parallel to the first biochip or the second biochip.

12. A method of hybridizing comprising:
disposing in a chamber a first biochip comprising a first probe surface, and a first barrier that is parallel to the first biochip;
hybridizing a biomolecule to the first biochip by providing a sample solution comprising the biomolecule between the first biochip and the first barrier;
disposing a second biochip comprising a second probe surface to be parallel to the first biochip,
wherein the first biochip and the second biochip are disposed such that a first probe surface of the first biochip and a second probe surface of the second biochip face each other;
removing the first barrier; and
performing hybridization in the second biochip by moving biomolecules which are bonded to the first biochip, to the second biochip.

13. The hybridization method of claim 12, wherein a target capture probe molecule is immobilized on the first probe surface of the first biochip, and a sample analyzing probe molecule is immobilized on the second probe surface of the second biochip.

14. The method of claim 13, wherein the target capture probe molecules and sample analyzing probe molecules bond to the same biomolecules and are formed on corresponding opposite areas of the first probe surface and the second probe surface.

15. The method of claim 12, wherein the performing hybridization in the second biochip comprises:
disposing a cathode near the first biochip and an anode near the second biochip to form an electrical field in a direction from the second biochip to the first biochip;
moving the biomolecules bonded to the first probe surface to the second probe surface using an electrophoresis method;
installing the first barrier again between the first biochip and the second biochip; and
catalyzing the hybridization in the second biochip by adjusting a reaction temperature.

16. The method of claim 12, wherein the performing hybridization in the second biochip comprises:
installing a plurality of second barriers between the first and second probe surfaces of the first and second biochips, wherein the plurality of second barriers are positioned to be perpendicular to the first and second probe surfaces of the first and second biochips so as to divide a space between the first biochip and the second biochip into a plurality of compartments;
cutting the biomolecules bonded to the first probe surface using a restriction enzyme; and
catalyzing the hybridization in the second biochip by adjusting a reaction temperature.

17. The method of claim 16, wherein restriction sites are formed in the biomolecules in the sample solution in advance.

18. The method of claim 12, further comprising, after performing the hybridization in the first biochip and removing the first barrier, connecting the terminal ends of the biomolecules bonded to probe molecules formed on the first probe surface with the terminal ends of the probe molecules formed on the first probe surface, using a ligase.

19. The method of claim 18, wherein the performing of the hybridization in the second biochip comprises:
adjusting a distance between the first biochip and the second biochip to be less than 1 μm; and
allowing the biomolecules to be denatured from the probe molecules formed on the first probe surface and then bonded to the probe molecules formed on the second probe surface by adjusting a reaction temperature,
wherein the terminal ends of the biomolecules are continuously connected to the terminal ends of the probe molecules formed on the first probe surface after being denatured from the probe molecules formed on the first probe surface.

20. The method of claim 12, further comprising, after performing the hybridization in the first biochip and before removing the first barrier, amplifying the biomolecules hybridized on the first biochip.

21. The method of claim 20, wherein the amplifying of the biomolecules is performed using a polymerase chain reaction (PCR) method and the method further comprises performing a fluorescent labeling process on the amplified biomolecules.

22. The method of claim 21, further comprises performing a fluorescent labeling process on the amplified biomolecules.
